(19) United States
(12) Patent Application Publication
(10) Pub. No.: US 2025/0072982 A1
(43) Pub. Date: Mar. 6, 2025

(54) MEDICAL ROBOTIC SYSTEM WITH ACTIVATION BUTTON AND ACTIVATION PROCEDURE DISPLAYED

(71) Applicant: B. Braun New Ventures GmbH, Freiburg im Breisgau (DE)

(72) Inventors: Tim Beyl, Freiburg im Breisgau (DE); Cathrin Senst, Freiburg (DE)

(21) Appl. No.: 18/822,712

(22) Filed: Sep. 3, 2024

(30) Foreign Application Priority Data
Sep. 5, 2023 (DE) 10 2023 123 838.8

(51) Int. Cl. A61B 34/30; A61B 34/00

(52) U.S. Cl. CPC A61B 34/30; A61B 34/25; A61B 2034/304; A61B 2034/305

(57) ABSTRACT

A medical robot system includes a robot with a movable robot arm connected to a robot base and a robot head connected to the robot arm. An end effector can be on the robot head or serve as a robot head. A control device can execute an action of the robot, in particular to control a movement of the robot and execute the movement as an action. The robot system further includes at least one touch display with a control unit. An activation button is visually displayed on the touch display. When the activation button is pressed as an input by a user, a control signal is sent to the robot control device via a signal connection to activate an action of the robot, such as a movement of the robot. The robot system can be implemented with an activation method, a computer-readable storage medium, and a computer program.

### CROSS-REFERENCE TO RELATED APPLICATIONS

This application claims priority under 35 U.S.C. § 119 to German Application No. 10 2023 838.8, filed on Sep. 5, 2023, the content of which is incorporated by reference herein in its entirety.

### FIELD

The present disclosure relates to a medical robot system for a surgical procedure on a patient, comprising: a robot having a movable robot arm connected to a robot base and a robot head terminally connected to the robot arm, in particular having an end effector on the robot head or as a robot head; and a robot control device adapted to perform an action of the robot, in particular to control a movement of the robot and to perform it as an action. The robot has various functions that can be activated, such as movement or manipulation with the end effector or visualization with a visualization unit as the end effector, and can also be deactivated accordingly. In addition, the present disclosure relates to an activation method and a computer-readable storage medium and a computer program.

### BACKGROUND

Current surgical robots either use hardware buttons, i.e., physical switches hardwired/wired to a control device and used to enable the movement of a robot or a robot-guided device, such as an end effector of the robot, or they use physical buttons that are evaluated by means of an electronic unit, which are then wired to a control device of the robot (robot controller) via a suitable communication protocol. All of these solutions require the placement of a physical device in the form of a switch within reach of the surgeon, which is used to operate the robot. Typically, such buttons are implemented as activation switches with either two positions (on-off; two-position switch) or three positions (off-on-off; three-position switch), whereby the button must be pressed in the two-position version and the button must be held in the middle in the three-position version.

Unfortunately, such solutions require additional components in the form of the physical switch and are also prone to errors, for example if a button gets stuck or jammed and continues to move the robot, which can lead to serious consequences in the event of an intervention. The buttons, for example, are also difficult to sterilize.

### SUMMARY

It is therefore the task of the present disclosure to avoid or at least reduce the disadvantages of the prior art and, in particular, to provide a medical robot system, an activation method, a computer-readable storage medium and a computer program which enables simple and safe use in order to activate and deactivate actions or functions of the robot.

A basic idea of the present disclosure thus provides for a touch display of a robot system which displays a visual activation switch or button and, when the button on the touch display is pressed, a control unit of the touch display requests activation of an action of the robot, such as starting a movement of the robot, and when the displayed activation switch is released, this action is then stopped or deactivated accordingly. For example, the surgeon can move the robot by pressing the touch display and, if another employee moves into the robot arm's range of motion, the robot can be stopped immediately when the activation button is released (i.e., when the finger is removed from the touch display). The control signal is sent to the robot via a signal connection between the control unit of the touch display and a robot control device, which controls the movement of the robot and allows different configurations of the robot arm in space, for example, and the action, in this example the movement, is activated and executed, and if no control signal arrives, the movement is stopped accordingly. In addition to this function of displaying the activation button, the touch display can also show other functions, for example, so that the activation function can be integrated without any other additional components such as a physical switch. A touch display with a flat surface can also be cleaned even better and a physical button can be prevented from getting stuck, for example because a cover cloth gets caught in a gap.

In other words, a (software-based) activation switch for a medical, in particular surgical, robot system is proposed that uses a single (signal) channel and yet enables safe operation of a surgical robot. The activation signal/release signal can be distributed/transmitted by real-time or non-real-time software and hardware.

In still other words, a medical robot system for a surgical procedure on a patient is provided, the robot system comprising: a robot having a movable robot arm connected to a robot base and a robot head terminally connected to the robot arm, in particular having an end effector on the robot head or as the robot head; a robot control device adapted to perform an action of the robot, in particular to control and perform as an action a movement of the robot, the robot system further comprising: (at least) a touch display/touch screen/touch screen/with a control unit adapted to visually display an activation button on the display and, upon pressing the activation button as an input by a user via a signal connection, such as a signal cable, to send a control signal/activation signal to the robot control device to activate an action of the robot, in particular to activate a movement of the medical robot. In particular, the touch display is arranged on the robot and is especially preferably attached or integrated on the robot head.

In still other words, in contrast to prior art solutions, an enable/activation button (software-based, so to speak) is used, which is displayed on a touchscreen or touchscreen-like device, or the touchscreen itself is used as an enable button. This means that existing touchscreens in the surgical area, which can also be sterile, can be used as activation buttons. Several activation buttons with different functions can also be implemented, such as the activation of a robot movement or a zero-space movement of a robot arm. Several such buttons can also be implemented on the same (one) touchscreen. In particular, a touchscreen can be provided in the sterile area, which is draped in particular, in combination with a control unit for the robot (robot control device) and a correspondingly adapted control unit, which releases or activates a robot movement. The touchscreen visually displays an activation/release button that can be pressed by the user and can also be visually adjusted further if necessary, for example so that it is displayed in green when the activation button is pressed and red when it is released, in order to intuitively display a current status.

Advantageous embodiments are explained in particular below.

According to one embodiment, the control unit can detect a binary input via the touch display of the two alternatives 1. press/key press—2. no press/no key press and only send a control signal to the robot control device when a key is pressed in order to activate the action and deactivate the action if no control signal is present. Preferably, the control unit that evaluates the touches on the touchscreen is adapted to transmit only the pressing (1) and releasing (0) of a button to the robot control device as a signal (1 or 0). The robot control device then starts the movement of the robot when the button is pressed and stops the movement when the button is released. The touchscreen is thus evaluated in real time and in a secure manner.

According to a further embodiment, the control unit can be adapted to send a heartbeat signal with a predetermined frequency as a control signal to the robot control device upon input by the user and the robot control device can be adapted to activate the action of the robot only upon receipt of the heartbeat signal with the predetermined frequency and to deactivate it otherwise. In other words, when the activation button is pressed, the touch signal is analyzed/evaluated by the control unit provided on the touchscreen and a so-called heartbeat signal or “keep-alive” signal or heartbeat signal is generated periodically, similar to a heartbeat. The heartbeat signal is evaluated by the robot control device. The robot control device expects the heartbeat signal at a certain frequency and would let the robot drive as long as the heartbeat signal is received properly. The destination for the robot to move to while the button is pressed can be calculated or provided by various sources such as a computer connected to the touchscreen, the robot control device itself, or another connected computer.

Preferably, a heartbeat signal with a predetermined fixed frequency, for example 10 Hz, can be generated when the button is pressed, whereby the robot control device is adapted to allow the movement as long as the heartbeat signal arrives within the assumed frequency. The movement stops as soon as the heartbeat signal stops.

Preferably, the control unit can be adapted to send a heartbeat signal with a predetermined first frequency as a control signal to the robot control device upon an input by the user and the robot control device can be adapted to expect a predetermined second frequency which is lower than the predetermined first frequency and to activate the action of the robot only upon receipt of the heartbeat signal with the lower predetermined second frequency and to deactivate it otherwise. In particular, the control device may be adapted to expect or request the heartbeat signal at a lower frequency as used and emitted by the activation switch. In this way, communication can be made more robust and systems that do not operate in real time or unreliable communication channels can be used to provide the signal. For example, the “activation button” or the control unit of the touch display (as transmitter) can provide a heartbeat signal with a frequency of 10 Hz, while the robot control device (as receiver) only receives it with a frequency of 1 Hz. When a signal is received, the receiver would expect the next signal within the next second. In this way, several signals can be missed on the activation button side or on the transmission line as long as at least one signal arrives or is present every second.

Preferably, the predetermined first frequency can be between 2 Hz and 100 Hz, in particular 10 Hz, and the predetermined lower second frequency can be an integer factor lower, in particular 1 Hz. The first frequency is therefore an integer multiple greater than the second frequency, which is expected to be about twice or four times or six times or eight times or ten times as great.

According to one embodiment, in addition to the heartbeat signal, when the visual activation button is released by the user or when a visually displayed stop button is pressed as input by the user, the control unit can be adapted to send a (special further signal) stop signal to the robot control device, which directly/immediately/in real time immediately deactivates the (activated) action, in particular stops a movement, without waiting for the heartbeat signal to end. Preferably, a heartbeat signal can also be combined with a start and a stop signal (i.e., start signal, in particular in the form of a starting heartbeat signal and a separate stop signal), so that the movement is stopped when the button is pressed for a stop and not only when the heartbeat signal is no longer received. This stop signal with a corresponding stop of the robot's action can be transmitted even faster than when the robot control device is waiting for the end of a frequency cycle, for example.

In particular, the touch display can have a pressure sensor for detecting the amplitude of a press by the user, in particular in the form of a touch screen based on a resistor or a capacitive infrared detection or a surface acoustic wave or a pcap or an optical recording or an acoustic pulse detection. Preferably, the touch display itself can be evaluated for touch by a sensor or a measuring system. For example, the control unit of the touch display can provide an analog or digital signal to a device to be controlled or to the robot control device in order to evaluate the touch interaction with the user. This can be done in particular with resistive, capacitive, infrared, acoustic surface wave, virtual, pcap, optical imaging, acoustic pulse recognition touchscreens.

In particular, the touch can be localized on the touchscreen itself, so that it is possible to evaluate not only that the screen was pressed, but also where and preferably how hard it was pressed. In particular, the control unit can be adapted to send a control signal only when a button is pressed within the displayed activation button.

Furthermore, the control unit can preferably be adapted to send the control signal to the robot control device only when an amplitude of a pressure above a predetermined minimum pressure and/or below a predetermined maximum pressure is detected, in particular to implement a three-position switch (on-off). Preferably, therefore, an activation switch or activation button with three positions can be implemented, which allows a force or pressure to be measured. A movement is permitted if the force or pressure is above a freely definable minimum limit value but below a freely definable maximum limit value. In other words, minimum pressure and/or maximum pressure on the touchscreen is required for activation via the touchscreen.

According to one embodiment, the robot system can have a redundant configuration of the data connection between the control unit of the touch display and the robot control device and send a two-channel control signal. In particular, the evaluation of the touch interaction can be implemented redundantly in order to provide a two-channel touch signal to the robot controller or a control system. This signal can be provided by one or more control devices or measuring systems for touchscreens.

According to a further embodiment, in addition to the activation, the control signal can also send a further parameter for the activation, in particular a parameter of a target or a target point for the robot or a parameter of a direction of movement or a parameter of a unique signal identifier. In other words, the heartbeat signal in particular can also transmit further information such as the target for the robot or a direction or a unique signal identifier.

Preferably, the user can select the activation signal himself, for example via a menu with corresponding activation signals shown on the touch display.

Preferably, the control unit can be adapted to select and send a suitable activation to be triggered depending on the surgical situation, for example depending on a current step of the operation, for example to move the robot head when activated in a first step of the operation and to activate a manipulation by the end effector, for example sclerotherapy, in a second step of the operation.

Preferably, several touch displays with displayed activation buttons can be used simultaneously, which are arranged in particular at different locations in the operating room. All control units of the different touch displays are connected to the central robot control device via data technology and each individual touch display enables an action such as a movement of the robot to be activated.

In particular, the activation switch can be configurable, for example in its visual display or an activation target.

According to a further embodiment, in addition to the touch display, the robot system can also have a further input unit, in particular in the form of a foot pedal or a physical switch, in order to activate the action of the robot, in particular also via the further input unit, with corresponding deactivation when the foot pedal is no longer pressed. Preferably, the touch display can have a further input unit in the form of a foot pedal, another touch screen, a physical button or switch as an input and can be supplemented by these to a certain extent. In complex scenarios, this means that touchscreen interaction cannot be the only input for generating an activation signal. For example, the user can make an activation input via the touch display or alternatively via a foot pedal, which is also connected via a signal.

Preferably, the touch display can be movably guided in a housing and, when the activation button is pressed as the input by the user, the touch display can be physically translatable in the housing, so that the touch display itself represents a physical switch and, in particular, the control unit only sends the control signal in an intermediate position between a minimum depression and a maximum depression in order to implement a three-position switch (off-on-off). In other words, the touch display itself can be a button (pressing the button when pressing the touch screen). It can have around two or three positions and can be implemented as a one or two-channel solution.

According to one embodiment, a further touch-sensitive surface can be arranged in front of or behind the touchscreen, in particular a second touchscreen, and a distance sensor can detect a distance between the touchscreen and the touch-sensitive surface and send the control unit the control signal for activation if the distance falls below a predetermined distance. Alternatively, in other words, the button may preferably not be a mechanical button, but a second touch display in front of the first touch display or another interaction technology arranged behind a touch display or in front of a touch display. Examples could be a distance measurement system between the touch display and a second touch display or between the touch-sensitive area and the screen, or a camera system that observes the touch display.

In particular, several activation modalities can be used in parallel to generate a redundant activation signal.

With regard to an activation method for a medical robot system for a surgical procedure on a patient with a robot, in particular a robot system according to the present disclosure, it comprises the steps of: Displaying a visual activation button on a touch display of the robot system; detecting a press of the displayed activation button as an input by a user and, upon detection of the press, sending a control signal from a control unit to a robot control device; receiving the control signal by the robot control device and, upon receipt, then activating an action of the robot by the robot control device.

With regard to a computer-readable storage medium and with regard to a computer program, the tasks are solved in each case in that the latter comprises instructions which, when executed by a computer, cause the computer to carry out the steps of the activation method according to the present disclosure.

It should be noted that the concept can also be used outside of surgery and can be integrated as an activation switch for other safety-critical or regulated applications in terms of a single fault-tolerant system, for example in a medical laboratory.

### BRIEF DESCRIPTION OF THE DRAWINGS

The present disclosure is explained in more detail below with reference to preferred embodiments with the aid of figures.

FIG. 1 is a schematic front view of a medical robot system comprising a robot and a touch display according to a preferred embodiment of the present disclosure; and

FIG. 2 is a flow chart of an activation process according to a preferred embodiment of the present disclosure.

The figures are schematic in nature and are only intended to aid understanding of the present disclosure.

### DETAILED DESCRIPTION

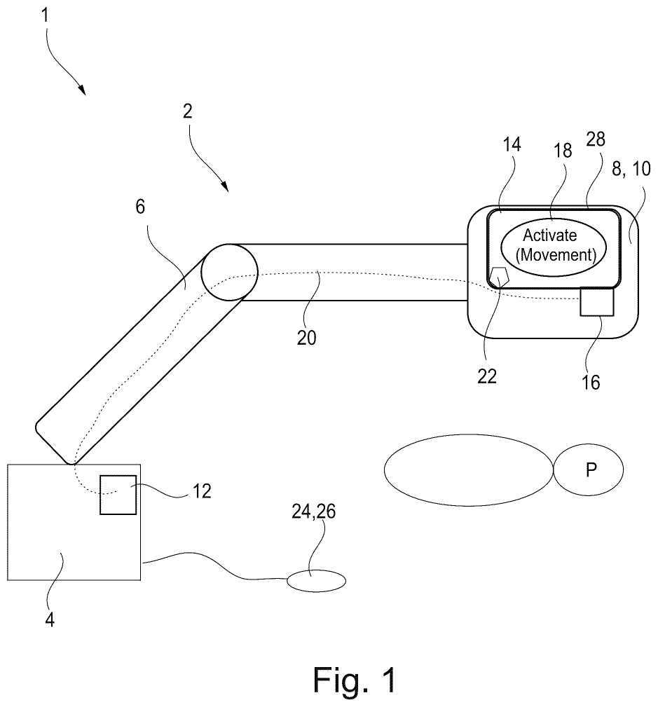

FIG. 1 shows a schematic view of a medical, in the present case a surgical robot system 1 (hereinafter referred to only as robot system) for a surgical intervention on a patient P according to a first preferred embodiment. The robot system 1 has a robot 2. The robot has a robot base 4 attached to a floor of an operating theater and a movable, multi-segment robot arm 6 connected to it as well as a robot head 8 connected to the end of the robot arm 6 with an end effector 10 as the robot head 8 in the form of a surgical microscope, the microscope head of which is integrated in the robot head with the optical system and the recording sensor. A robot control device 12 provided in the robot base 4 is adapted to perform a plurality of different actions of the robot, in which as a subset a movement of the robot is one action and a change of the visualization parameters is another different action.

A touch display 14 is rigidly attached to the robot head, which has a control unit 16 as the central data processing unit. The touch display 14 is adapted to visually display (in a display mode) an activation button 18 on the touch display 14 or the display. Furthermore, when the activation button 18 is pressed, the touch display detects this as an input by a user and is adapted to send a control signal to the robot control device 12 via a wired robot-internal signal connection 20, which is shown schematically as a dashed line, in order to activate an action of the robot 2, in particular to activate a movement of the medical robot 2. As soon as the activation button is no longer pressed and no control signal is sent, the robot control device 12 stops the action immediately or deactivates it.

In this way, the touch display 14 already provided in the robot system 1 is used to provide a non-physical activation button 18 which the user can press, in particular to move the robot 2, for example to a target point currently set according to the operation plan for optical aiming. In addition, other functions can also be controlled via the touch display, so that the touch display provides different functional modalities centrally without the need for additional buttons. The touch display can also be draped particularly well in sterile conditions for use in a surgical procedure.

In this embodiment, the control unit 16 is adapted to send a heartbeat signal at a predetermined first frequency as a control signal to the robot control device 12 upon input by the user, and the robot control device 12 is in turn adapted to expect a predetermined second frequency that is lower than the predetermined first frequency and to activate the action of the robot 2 upon receipt of the heartbeat signal at the lower predetermined second frequency and to deactivate it otherwise. This heartbeat signal ensures that the robot only actually moves when the user presses the activation button 18 shown on the touch display 14 and provides a veritable safety function. If, for example, due to a software error in the control unit 16, a signal is sent, but not at the specified frequency, the robot control device detects this and thus also an error and stops or deactivates the movement as an action. In this embodiment, the control unit 16 transmits a heartbeat signal at a frequency of 10 Hz, i.e., ten signals per second, whereby the robot control device 12 expects only 1 Hz. The nine out of ten signals are not relevant as long as at least one signal is sent evenly every second. This prevents any latency.

In this embodiment shown in FIG. 1, the robot system 1 also has a further input unit 24 in the form of a foot pedal 26 in addition to the touch display 14 as an input option. Alternatively, a physical switch (not shown) can also be provided, for example on a wall of the operating theater. In this way, the action of robot 2 can also be activated via the additional input unit.

The touch display 14 has a pressure sensor 22, which measures manual pressure on the touch display. The measurement is therefore not binary, but the amplitude of a press of the finger on the touch display 14 is determined. As a further safety precaution, the control unit 16 is adapted to send the control signal only when a minimum pressure is applied to the activation switch 18. Furthermore, the control unit 16 is also adapted to send the control signal only below a maximum pressure, for example to prevent the robot from moving if the touch display 14 collides with an object in the operating room.

It should also be mentioned that the present robot system 1 also has the advantage that the control unit can also be adapted so that a control signal is only sent after a minimum time of pressing the activation button 18 in order to avoid unintentional triggering, for example in the event of a brief touch when passing by.

Furthermore, in an optional embodiment, the touch display 14 has a housing 28 and is slidably mounted in this housing so that the touch display 14 itself can be pressed as a button.

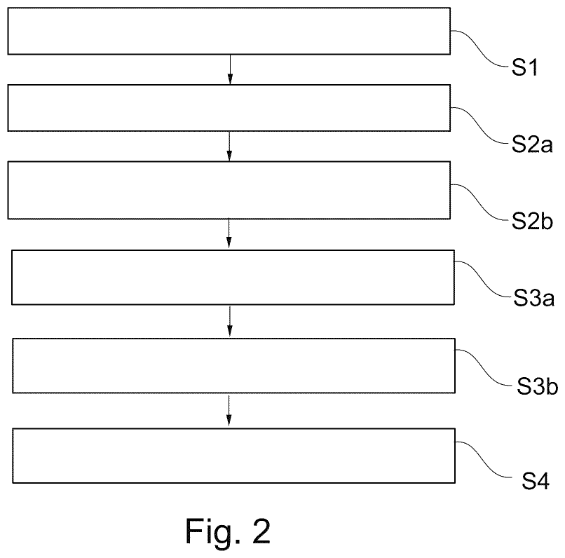

FIG. 2 shows in a flowchart an activation method according to a preferred embodiment for a medical robot system 1 according to the present disclosure.

In a first step S1, a visual activation button 18 is displayed on the touch display 14 of the medical robot system 1

In a subsequent step S2a, a press of the displayed activation button 18 is continuously detected as an input by a user.

If an input is detected, only then and only during the input in a step S2b is a control signal sent from a control unit 16 to the robot control device 12 in order to activate an action of the robot 2.

The robot control device 12 continuously listens (or expects) for a control signal and, at a step S3a of receiving the control signal by the robot control device 12 (i.e., when the activation signal actually arrives), a currently selected action of the robot 2 is activated by the robot control device 12 in a step S3b and this action is executed. This activation only takes place as long as the control signal remains on and arrives.

In step S4, the action is deactivated as soon as no control signal or, in this version, no activation signal is received.

In particular, the activation method can be used to start and stop a movement of the robot 2 by means of a touch display, namely for as long as the user keeps the displayed activation button 18 pressed.

### LIST OF REFERENCE SIGNS

1 Medical robot system
2 Robot
4 Robot base
6 Robot arm
8 Robot head
10 End effector
12 Robot control device
14 Touch display
16 Control unit
18 Activation button
20 Signal connection
22 Pressure sensor
24 Additional input unit
26 Foot pedal
28 Housing
P Patient
S1 Step display Activation button on touch display
S2a Step Detecting a press of the activation button
S2b Step Sending the control signal
S3a Step Receiving the control signal
S3b Step Activating the robot's action

1. A medical robot system for a surgical procedure on a patient, the medical robot system comprising:
a robot with a robot arm connected to a robot base and a robot head connected at an end to the robot arm;
a robot control device adapted to execute an action of the robot; and
at least one touch display with a control unit adapted to visually display an activation button on the at least one touch display,
the activation button being pressable by a user as an input to send a control signal to the robot control device via a signal connection in order to activate an action of the robot.

2. The medical robot system according to claim 1, wherein the control unit is configured to:
detect when the activation button is pressed via the at least one touch display,
detect when the activation button is not pressed,
send the control signal to the robot control device to activate the action only when the activation button is pressed, and
deactivate the action when no control signal is present.

3. The medical robot system according to claim 1, wherein:
the control unit is adapted to send a heartbeat signal with a predetermined frequency as a control signal to the robot control device upon an input by the user, and
the robot control device is adapted to activate the action of the robot only upon receipt of the heartbeat signal with the predetermined frequency and to deactivate the action otherwise.

4. The medical robot system according to claim 1, wherein:
the control unit is adapted to send a heartbeat signal with a predetermined first frequency as a control signal to the robot control device upon an input by the user, and
the robot control device is adapted to:
expect a predetermined second frequency, which is lower than the predetermined first frequency,
activate the action of the robot when the robot control device receives a heartbeat signal with the predetermined second frequency, and
deactivate the action otherwise.

5. The medical robot system according to claim 4, wherein the predetermined first frequency is between 2 Hz and 100 Hz, and the predetermined second frequency is lower than the predetermined first frequency by an integer factor.

6. The medical robot system according to claim 5, wherein the predetermined second frequency is lower than the predetermined first frequency by 1 Hz.

7. The medical robot system according to claim 4, wherein, in addition to the heartbeat signal, when the visual activation button is released by the user or when a visually displayed stop button is pressed as input by the user, the control unit is adapted to send a stop signal to the robot control device, which directly deactivates the action without waiting for an end of the heartbeat signal.

8. The medical robot system according to claim 1, wherein the at least one touch display has a pressure sensor for detecting an amplitude of pressure when pressed by the user.

9. The medical robot system according to claim 1, wherein the at least one touch display comprises a touch screen that detects the amplitude of pressure based on a resistance or a capacitive infrared detection or a surface acoustic wave or a pcap or an optical recording or an acoustic pulse detection.

10. The medical robot system according to claim 9, further comprising a touch-sensitive surface arranged in front of or behind the touch screen, wherein a distance sensor detects a distance between the touch screen and the touch-sensitive surface and sends the control signal for activation to the control unit when the distance falls below a predetermined distance.

11. The medical robot system according to claim 9, wherein the control unit is adapted to send the control signal to the robot control device only when the amplitude of pressure is above a predetermined minimum pressure and/or below a predetermined maximum pressure.

12. The medical robot system according to claim 9, further comprising a redundant configuration of a data connection between the control unit and the robot control device, wherein the control signal is a two-channel control signal.

13. The medical robot system according to claim 1, wherein:
the control signal also transmits a further parameter for the activation, and
the robot control device processes and activates the further parameter and controls an action based on the parameter.

14. The medical robot system according to claim 13, wherein the further parameter comprises a target point for the robot, a direction of movement, or a unique signal identifier.

15. The medical robot system according to claim 1, further comprising a further input unit to carry out an activation of the action of the robot.

16. The medical robot system according to claim 15, wherein the further input unit comprises a foot pedal or a physical switch.

17. The medical robot system according to claim 1, wherein the at least one touch display is movably guided in a housing and, when the activation button is pressed, the at least one touch display is physically displaceable translationally in the housing, so that the at least one touch display represents a physical switch.

18. The medical robot system according to claim 17, wherein the control unit is configured to only transmit the control signal in an intermediate position between a minimum depression and a maximum depression in order to implement a three-position switch.

19. An activation method for a medical robot system according to claim 1, the activation method comprising the steps of:
displaying the activation button on the at least one touch display of the medical robot system;
detecting when the activation button is pressed by a user as the input;
sending the control signal from the control unit to the robot control device upon detecting when the activation button is pressed;
receiving the control signal by the robot control device; and
activating the action of the robot by the robot control device when the robot control device receives the control signal.

20. A computer-readable storage medium comprising instructions which, when executed by a computer, cause the computer to perform the activation method of claim 19.
